SEQUENCE LISTING (4 sequences)

SEQ ID NO: 1 - DNA, 1742 bp, Amebophrya sp.
  source 1..1742: mol_type = genomic DNA; note = OSK2020
tagtcatatgcttgtctcaaagattaagccatgcatgtctcagtatgagtctctatacggcgaaactgcgaatggctcattaaaacagttttaatctatttgatggccgcttgtcatatatggataactgtggaaattctagagctaatacatgcgtacagacccgacttcgcggaacggtcgtgcttattagatacagaaccaatacctccgggttgttggatgactcatagtaagtgaacggagcgcattggcatgagccggtgctagatcattcgagattctgacctatcagctttcgacggtagtgtattggactaccgtggcattgacgggtaacggggaattagggttcgattccggagagggagcccgagaaacggctaccacatctaaggaaggcagcaggcgcgcaaattacccaatcctgatacagggaggtagtgacaagaagtaacaaagcaagtgtctgagacattttgctattggaatgagtagaatttaaatcccttttcgactatcaattggagggcaagtctggtgccagcagccgcggtaattccagctccaatagcgtatattaaagttgttgcggttaaaaagctcgtagttggatttctgtggtaggcagccggtccacactatgtgtgagtatctggttcgactactgcatcctcctggagaacgttggtgcgcttcgctgcgtggcacggtattcaggacttttactttgaggaaattagagtgttcacggcaggcgatcgccttgaatatattagcatggaataatgatataggatcttcgtcctattttgttggtttctaggacgaggataatgattaatagggataattgggggcattcgtattaacacgtcagaggtggaattcttggattgtgttacgacgaactactgcgaaagcatttgccaaggatgttttcattgatcaagaacgaaagtaaggggatcgaagacgattagataccgtcgtagtctttaccataaactatgccgactagagattggaggtcgttctttgtatgaccccttcagcaccttatgagaaatcaaagtttttgggttccggggggagtatggtcgcaaggctggaacttaaaggaattgacggaagggcaccaccaggagtggagcctgcggcttaatttgactcaacacgggaaaactcaccaggtccagacatagtaaggattgacagattgatagctctttcttgattctatgggtggtggtgcatggccgttcttagttggtggagtgatttgtcaggttaattccgttaacgaacgagaccttatcctgctaaatagcgcagcgtatcttcggatatgcagtttgcttcttagagggactttgtgtttttcagcgcaaggaagtttgaggcaataacaggtctgtgatgcccttagatgttctgggctgcacgcgcgctacactgatgcgttcagcgagtttataaccttgcccggtagggttgggtaatcttctgaaagcgcatcgtgatggggatagattattgtaattattaatcttcaacgaggaattcctagtaagcgtgaatcatcagttcacgctgattacgtccctgccctttgtacacaccgcccgtcgctcctaccgattgagtgatccggtgaataattcggacctgcgagtcttgcggttcgctgcttggcctgtgggaaagtttagtgaaccttatcacttagaggaaggagaagt

SEQ ID NO: 2 - DNA, 759 bp, Amebophrya sp.
  source 1..759: mol_type = genomic DNA; note = OSK2020
taagtaaggccaggaaaagaaaatgaatatgactcccttagtaatggcgaatgaaccgggacgagctcaacttgtaaatccgcggcttgctgcggagttgtagtctatagagctatcactagtgcgtgcccccgtctaactctttttggaaagaaggggcatagagggtgagactcccgtctggtggcgggcgcgcgcggtcagtgtggcttcttcgagtcgcgtactttggaaatggtgcgctaagcggggggtaattttcctccaaagctaagtatgggactgggaccgatagaaaacaagtaccgtgagggaaaggtgaaaaggactttggaaagagagttaaaagtgcctgaaatcgctaagagggaagcgaatgaaaccagtgacttctcgggagattgctgccggctatagtgaaaaaaccgtacttcgggcgggaatgatctggccgtcggtggtgcttctcctgttaggcgtcagcgtaagttgggcgcggaagtataagcgctttctaatgtaggttctctgctgggttcctgcatggagggttgcggcttccgtctcgacttgggattgcgggtggctcgcaatgtgctctcatcctagcgacaccgttgcggaccttcgggtgcgtaatggcgagtgtggaggcggtacaatcctgtcactccatgacgttggctaagtggttttattctacccgtcttgaaacacggaccaaggagtcgaactcatacgcgagtatgcgggtgtcaa

SEQ ID NO: 3 - DNA, 760 bp, Amebophrya sp.
  source 1..760: mol_type = genomic DNA; note = HMN2020
taagcggaggaaaagaaaatgaacatgattcccttagtaagggcgactgaaccgggaagagctcaaaatgtaaaaccaagacttgtcttggagttgtaaccttgagatccatcaccagcgcgagcctctgcgtaagtctttttggaaaaaagcgtcaaagagggtgagaaccccgtccacgtggagaggtagtgcatccggtgtggtgtcgtagagtcgcgcactttggacttggtgtgcaaaacgggaggtaattgtcttctaaagctaaatacagacttgagaccgatagcaaacaagtaccgtgagggaaagatgaaaaggactttggaaagagagttaaaagtgcctgaaatcgctaagagagaagcgaatgaagtcagtttctttaccagagattggggcaggctaaggtgaataaccataccgcaagggtgggcatttgatctggccgtctgttctgtttctttggtttagttgtcatcgtatgctgggtgctcgaggattatcgttgtgctccgttgttctgctgcggtggttcgcgaggtacttctgagactcttgccccggcataggtttgcgctcagcttctatttggccataagcagcttgaaggttcggtggacttctgaagtgctcagttctgatggctgatgaggtcttatgtgctgtcccacgacgatgaccaaatggctttattctacccgtcttgaaacacggaccaaggagtcgacctctatcgcaagtgtgcgggtggcaa

SEQ ID NO: 4 - DNA, 759 bp, Amebophrya sp.
  source 1..759: mol_type = genomic DNA; note = OSK2020
taagtaaggccaggaaaagaaaatgaatatgactcccttagtaatggcgaatgaaccgggacgagctcaacttgtaaatccgcggcttgctgcggagttgtagtctatagagctatcactagtgcgtgcccccgtctaactctttttggaaagaaggggcatagagggtgagactcccgtctggtggcgggcgcgcgcggtcagtgtggcttcttcgagtcgcgtactttggaaatggtgcgctaagcggggggtaattttcctccaaagctaagtatgggactgggaccgatagaaaacaagtaccgtgagggaaaggtgaaaaggactttggaaagagagttaaaagtgcctgaaatcgctaagagggaagcgaatgaaaccagtgacttctcgggagattgctgccggctatagtgaaaaaaccgtacttcgggcgggaatgatctggccgtcggtggtgcttctcctgttaggcgtcagcgtaagttgggcgcggaagtataagcgctttctaatgtgggttctctgctgggttcctgcatggagggttgcggcttccgtctcgacttgggattgcgggtggctcgcaatgtgctctcatcctagcgacaccgttgcggaccttcgggtgcgtaatggcgagtgtggaggcggtacaatcctgtcactccatgacgttggctaagtggttttattctacccgtcttgaaacacggaccaaggagtcgaactcatacgcgagtatgcgggtgtcaa

(19) United States
(12) Patent Application Publication
(10) Pub. No.: US 2025/0324981 A1
(43) Pub. Date: Oct. 23, 2025

(54) PARASITIC DINOFLAGELLATE BELONGING TO GENUS AMOEBOPHRYA

(71) Applicants: TOHOKU UNIVERSITY, Miyagi (JP); RESEARCH INSTITUTE OF ENVIRONMENT, AGRICULTURE AND FISHERIES, OSAKA PREFECTURE, Osaka (JP)

(72) Inventors: Goh NISHITANI, Miyagi (JP); Keigo YAMAMOTO, Osaka (JP); Masaki NAKAJIMA, Osaka (JP)

(73) Assignees: TOHOKU UNIVERSITY, Miyagi (JP); RESEARCH INSTITUTE OF ENVIRONMENT, AGRICULTURE AND FISHERIES, OSAKA PREFECTURE, Osaka (JP)

(21) Appl. No.: 18/867,132

(22) PCT Filed: May 18, 2023
(86) PCT No.: PCT/JP2023/018620

§ 371 Date: Nov. 19, 2024

(30) Foreign Application Priority Data
May 20, 2022 (JP) 2022-083308

(51) Int. Cl. A01N 65/03; A01P 13/02

(52) U.S. Cl. CPC A01N 65/03; A01P 13/02

(57) ABSTRACT

An object is to provide parasitic dinoflagellates of the genus Amoebophrya that are effective in the prevention of red tide derived from dinoflagellates of the genus Karenia. Provided are a parasitic dinoflagellate of the genus Amoebophrya having the ability to parasitize a dinoflagellate of the genus Karenia, a composition and a microbial pesticide, each containing a parasitic dinoflagellate of the genus Amoebophrya, a method for obtaining a parasitic dinoflagellate of the genus Amoebophrya, and a method for inhibiting the proliferation of dinoflagellates of the genus Karenia.

### TECHNICAL FIELD

The present invention relates to parasitic dinoflagellates of the genus Amoebophrya.

### BACKGROUND ART

Red tide due to plankton overgrowth is known to cause oxygen deficiency in seawater and cause enormous damage to aquaculture. One type of plankton that causes red tide is the dinoflagellates of the genus Karenia, and the damage caused by the plankton has been confirmed in coastal areas around the world.

NPL 1 reports that Flavobacterium sp. isolated from Ise Bay has an algicidal effect on dinoflagellates of the genus Karenia. However, since Flavobacterium sp. is a bacterium, it has not yet been put to practical use as a red-tide control agent due to the potential impact on organisms other than the dinoflagellates of the genus Karenia. So far, no effective means of exterminating the dinoflagellates of the genus Karenia has been found.

### CITATION LIST

### Non-patent Literature

NPL 1: Nippon Suisan Gakkaishi 70 (4), 537-541 (2004)

### SUMMARY OF INVENTION

### Technical Problem

An object of the present invention is to provide parasitic dinoflagellates of the genus Amoebophrya effective in preventing red tide derived from dinoflagellates of the genus Karenia.

### Solution to Problem

In order to solve the problem described above, the present inventors conducted extensive research and found that a specific parasitic dinoflagellate of the genus Amoebophrya has the ability to parasitize and kill the dinoflagellates of the genus Karenia.

The present invention was completed based on the finding and includes the subject matter of a wide range of aspects described below.

### Item 1

A parasitic dinoflagellate of the genus Amoebophrya, having the ability to parasitize a dinoflagellate of the genus Karenia.

### Item 2

A parasitic dinoflagellate of the genus Amoebophrya, comprising one of the following nucleic acid molecules (1) to (12):
(1) a nucleic acid molecule containing the base sequence of SEQ ID NO: 1,
(2) a nucleic acid molecule containing a base sequence having one or several bases substituted, added, or deleted in the base sequence of SEQ ID NO: 1,
(3) a nucleic acid molecule containing a base sequence having at least 90% identity with the base sequence of SEQ ID NO: 1,
(4) a nucleic acid molecule containing a base sequence that hybridizes with a base sequence complementary to the base sequence of SEQ ID NO: 1 under stringent conditions,
(5) a nucleic acid molecule containing the base sequence of SEQ ID NO: 2,
(6) a nucleic acid molecule containing a base sequence having one or several bases substituted, added, or deleted in the base sequence of SEQ ID NO: 2,
(7) a nucleic acid molecule containing a base sequence having at least 90% identity with the base sequence of SEQ ID NO: 2,
(8) a nucleic acid molecule containing a base sequence that hybridizes with a base sequence complementary to the base sequence of SEQ ID NO: 2 under stringent conditions,
(9) a nucleic acid molecule containing the base sequence of SEQ ID NO: 3,
(10) a nucleic acid molecule containing a base sequence having one or several bases substituted, added, or deleted in the base sequence of SEQ ID NO: 3,
(11) a nucleic acid molecule containing a base sequence having at least 90% identity with the base sequence of SEQ ID NO: 3, and
(12) a nucleic acid molecule containing a base sequence that hybridizes with a base sequence complementary to the base sequence of SEQ ID NO: 3 under stringent conditions.

### Item 3

The parasitic dinoflagellate according to Item 1, having an ability to kill a dinoflagellate of the genus Karenia.

### Item 4

The parasitic dinoflagellate according to Item 1, having an ability to inhibit the proliferation of a dinoflagellate of the genus Karenia.

### Item 5

A composition comprising the parasitic dinoflagellate of any one of Items 1 to 4.

### Item 6

The composition according to Item 5, which is for use in the prevention of red tide.

### Item 7

A microbial pesticide comprising the parasitic dinoflagellate of any one of Items 1 to 4.

### Item 8

A method for obtaining a parasitic dinoflagellate of the genus Amoebophrya, comprising the step of inoculating a dinoflagellate of the genus Karenia infected with a parasitic dinoflagellate of the genus Amoebophrya into a medium in which an uninfected dinoflagellate of the genus Karenia is cultured.

### Item 9

A parasitic dinoflagellate of the genus Amoebophrya obtained by the method of Item 8.

### Item 10

A method for inhibiting the proliferation of algae of the genus Karenia, comprising the step of allowing a parasitic dinoflagellate of the genus Amoebophrya to parasitize a dinoflagellate of the genus Karenia.

### Advantageous Effects of Invention

The present invention provides parasitic dinoflagellates of the genus Amoebophrya having the ability to parasitize dinoflagellates of the genus Karenia. The present invention also provides a microbial pesticide using parasitic dinoflagellates and a method for preventing red tide by using parasitic dinoflagellates.

### BRIEF DESCRIPTION OF DRAWINGS

FIG. 1 shows that Amoebophrya sp OSK2020, a parasitic dinoflagellate of the genus Amoebophrya, parasitizes Karenia mikimotoi, a dinoflagellate of the genus Karenia. The scale bars in FIG. 1 all indicate 20 μm. FIG. 1A: an image of uninfected Karenia mikimotoi observed with an optical microscope. FIG. 1B: an image of uninfected Karenia mikimotoi observed with a fluorescence microscope. FIG. 1C: an image of Karenia mikimotoi in the early stage of infection observed with an optical microscope. FIG. 1D: an image of Karenia mikimotoi in the early stage of infection observed with a fluorescence microscope. FIG. 1E: an image of Karenia mikimotoi in the middle stage of infection observed with an optical microscope. FIG. 1F: an image of Karenia mikimotoi in the middle stage of infection observed with a fluorescence microscope. FIG. 1G: an image of Karenia mikimotoi in the late stage of infection observed with an optical microscope. FIG. 1H: an image of Karenia mikimotoi in the late stage of infection observed with a fluorescence microscope. FIG. 1I: an image observed with an optical microscope when Karenia mikimotoi was destroyed and OSK2020 was released. FIG. 1J: an image observed with a fluorescence microscope when Karenia mikimotoi was destroyed and OSK2020 was released. FIG. 2A shows the experiment results of infection of Karenia mikimotoi with Amoebophrya sp. OSK2020 in Example 1. FIG.

2A shows the changes in cell density of Karenia mikimotoi over time when Amoebophrya sp. OSK2020 was added to the culture medium of Karenia mikimotoi at a point of 0 hours in time from the start of culture (treatment) and when Amoebophrya sp. OSK2020 was not added (control).

FIG. 2B shows the experiment results of infection of Karenia mikimotoi with Amoebophrya sp. OSK2020 in Example 1. FIG. 2B shows the infection rate (line graph) and infection state (bar graph) of Karenia mikimotoi when Amoebophrya sp. OSK2020 was added to the culture medium of Karenia mikimotoi at a point of 0 hours in time from the start of culture.

FIG. 2C shows the experiment results of infection of Karenia mikimotoi with Amoebophrya sp. OSK2020 in Example 1. FIG. 2C shows the change in cell density of Amoebophrya sp. OSK2020 over time when Amoebophrya sp. OSK2020 was added to the culture medium of Karenia mikimotoi at a point of 0 hours in time from the start of culture.

FIG. 3 shows the results of molecular phylogenetic analysis of parasitic organisms by using nucleus 18S rDNA.

FIG. 4 shows the results of molecular phylogenetic analysis of parasitic organisms by using nucleus 28S rDNA.

FIG. 5 shows the number of days a host remained when the host infected with a parasite was stored at low temperatures (14° C., 12° C., and 10° C.) in Example 7 (control: 20° C.). When the infected host was stored at an optimum temperature of about 20° C., the number of infected host cells decreased with the number of days, and the infected host cells disappeared completely after a certain period of time (10 to 20 days). However, when the infected host was stored at low temperatures, the number of days until extinction was extended.

FIG. 6 shows the number of days a host remained when the host infected with a parasite was stored at low light intensities (20 μmol photons m⁻² s⁻¹ and 5 μmol photons m⁻² s⁻¹) (control: 100 μmol photons m⁻² s⁻¹) in Example 8. When the infected host was stored at about 100 μmol photons m⁻² s⁻¹, the number of infected host cells decreased with the number of days, and the infected host cells disappeared completely after a certain period of time (10 to 20 days). However, when the infected host was stored at low light intensities, the number of days until extinction was extended to a maximum of 90 days.

### DESCRIPTION OF EMBODIMENTS

In the present specification, singular nouns (with “a,” “an,” “the,” etc.) include both the singular and the plural unless otherwise clearly stated in the present specification or if a clear contradiction is acknowledged in the context. In the specification, the term “comprising” is a concept that encompasses both consisting essentially of and consisting of.

In an embodiment, the present invention provides parasitic dinoflagellates of the genus Amoebophrya. In the embodiment, the parasitic dinoflagellates of the genus Amoebophrya of the present invention have the ability to parasitize dinoflagellates of the genus Karenia.

Examples of dinoflagellates of the genus Karenia include Karenia mikimotoi, Karenia papilionacea, and Karenia selliformis, with Karenia mikimotoi and Karenia papilionacea being preferred.

As used in the present specification, “having the ability to parasitize” means that parasitism is confirmed in at least one cell of dinoflagellates of the genus Karenia in co-culture of parasitic dinoflagellates of the genus Amoebophrya with dinoflagellates of the genus Karenia. Whether dinoflagellates of the genus Karenia are infected (parasitized) with dinoflagellates of the genus Amoebophrya can be confirmed, for example, according to the method described in the Examples given below.

The conditions for co-culture are not particularly limited. For example, a common medium for culturing marine microalgae is used for the medium. A specific example is Daigo's IMK medium (manufactured by Nihon Pharmaceutical Co., Ltd.). The culture temperature is, for example, but not particularly limited to, 15 to 30° C., and preferably 20 to 25° C. The light intensity is, for example, but not particularly limited to, 10 to 500 μmol photons m⁻² s⁻¹, and preferably 50 to 200 μmol photons m⁻² s⁻¹. The light and dark cycle is also, for example, but not particularly limited to, the following: 20 hours of light and 4 hours of darkness to 4 hours of light and 20 hours of darkness; and preferably the following: 12 hours of light and 12 hours of darkness to 14 hours of light and 10 hours of darkness. The concentration of dinoflagellates of the genus Karenia at the start of co-culture is also not particularly limited; the concentration of dinoflagellates of the genus Karenia is, for example, 500 to 3000 cells mL⁻¹, and preferably 1000 to 2000 cells mL⁻¹. The concentration of parasitic dinoflagellates of the genus Amoebophrya is also not particularly limited. For example, the concentration of parasitic dinoflagellates of the genus Amoebophrya may be 5000 to 30000 cells mL⁻¹, and preferably 10000 to 20000 cells mL⁻¹.

In co-culture of the parasitic dinoflagellates of the genus Amoebophrya of the present invention with dinoflagellates of the genus Karenia (e.g., co-culture for 12 hours or more according to the method described in Example 1), the infection rate of the dinoflagellates of the genus Karenia is not particularly limited as long as it exceeds 0%; however, the infection rate is preferably 10% or more, more preferably 50% or more, and even more preferably 90% or more.

The parasitic dinoflagellates of the genus Amoebophrya of the present invention have the ability to inhibit the proliferation of dinoflagellates of the genus Karenia when co-cultured with the dinoflagellates of the genus Karenia. This is because the parasitic dinoflagellates of the genus Amoebophrya of the present invention have the ability to kill the dinoflagellates of the genus Karenia after parasitizing the dinoflagellates. In co-culture of the parasitic dinoflagellates of the genus Amoebophrya of the present invention with dinoflagellates of the genus Karenia (e.g., co-culture for 12 hours or more according to the method described in Example 1), the parasitic dinoflagellates of the genus Amoebophrya of the present invention preferably reduce 10% or more, more preferably 50% or more, and even more preferably 90% or more of the dinoflagellates of the genus Karenia as compared to before co-culture.

The parasitic dinoflagellates of the genus Amoebophrya of the present invention have the ability to kill the dinoflagellates of the genus Karenia after parasitizing the dinoflagellates of the genus Karenia. In co-culture of the parasitic dinoflagellates of the genus Amoebophrya of the present invention with dinoflagellates of the genus Karenia (e.g., co-culture for 12 hours or more according to the method described in Example 1), the parasitic dinoflagellates of the genus Amoebophrya of the present invention preferably kill 10% or more, more preferably 50% or more, and even more preferably 90% or more of the dinoflagellates of the genus Karenia.

In addition, when the parasitic dinoflagellates of the genus Amoebophrya of the present invention are sprayed over the sea surface or into seawater, for example, as a microbial pesticide, it is preferred that the parasitic dinoflagellates of the genus Amoebophrya of the present invention have the ability to specifically parasitize dinoflagellates of the genus Karenia for the sake of little concern about infection of an unspecified number of organisms and a high degree of safety. Specifically, when the parasitic dinoflagellates of the genus Amoebophrya of the present invention are co-cultured with an organism other than dinoflagellates of the genus Karenia, it is preferred that no infection be confirmed in the organism.

The parasitic dinoflagellates of the genus Amoebophrya of the present invention preferably have one of the following nucleic acid molecules (1) to (12):
(1) a nucleic acid molecule containing the base sequence of SEQ ID NO: 1,
(2) a nucleic acid molecule containing a base sequence having one or several bases substituted, added, or deleted in the base sequence of SEQ ID NO: 1,
(3) a nucleic acid molecule containing a base sequence having at least 90% identity with the base sequence of SEQ ID NO: 1,
(4) a nucleic acid molecule containing a base sequence that hybridizes with a base sequence complementary to the base sequence of SEQ ID NO: 1 under stringent conditions,
(5) a nucleic acid molecule containing the base sequence of SEQ ID NO: 2,
(6) a nucleic acid molecule containing a base sequence having one or several bases substituted, added, or deleted in the base sequence of SEQ ID NO: 2,
(7) a nucleic acid molecule containing a base sequence having at least 90% identity with the base sequence of SEQ ID NO: 2,
(8) a nucleic acid molecule containing a base sequence that hybridizes with a base sequence complementary to the base sequence of SEQ ID NO: 2 under stringent conditions,
(9) a nucleic acid molecule containing the base sequence of SEQ ID NO: 3,
(10) a nucleic acid molecule containing a base sequence having one or several bases substituted, added, or deleted in the base sequence of SEQ ID NO: 3,
(11) a nucleic acid molecule containing a base sequence having at least 90% identity with the base sequence of SEQ ID NO: 3, and
(12) a nucleic acid molecule containing a base sequence that hybridizes with a base sequence complementary to the base sequence of SEQ ID NO: 3 under stringent conditions.

In the present specification, the phrases “containing the base sequence” and “containing a base sequence” include an aspect in which the base sequence is contained as part of the full-length base sequence to the extent that the ability to parasitize dinoflagellate of the genus Karenia is not impaired, and an aspect in which the full-length base sequence essentially consists of the base sequence.

In (1) above, the nucleic acid molecule encompasses a nucleic acid molecule containing the base sequence of SEQ ID NO: 1 enabling the parasitic dinoflagellates of the genus Amoebophrya having the nucleic acid molecule to have the ability to parasitize dinoflagellates of the genus Karenia.

In (2) above, the number of one or several bases to be substituted, deleted, added, or inserted is not particularly limited as long as the number is an integer of 1 or more. For example, the number of one or several bases may be 1 to about several dozen, preferably about 1 to 30, more preferably about 1 to 15, even more preferably about 1 to 10, and particularly preferably about 1 to 5. The nucleic acid molecule encompasses a nucleic acid molecule containing a base sequence having one or several bases substituted, added, or deleted in the base sequence of SEQ ID NO: 1 enabling the parasitic dinoflagellates of the genus Amoebophrya having the nucleic acid molecule to have the ability to parasitize dinoflagellates of the genus Karenia.

In an aspect, the number of one or more bases to be substituted, added, or deleted may be about 1 to 174 so that the identity of the base sequence with the base sequence of SEQ ID NO: 1 is at least 90%, preferably about 1 to 87 so that the identity is at least 95%, more preferably about 1 to 35 so that the identity is at least 98%, and particularly preferably about 1 to 17 so that the identity is at least 99%.

In (3) above, the identity of the base sequence may be at least 90%, preferably at least 95%, more preferably at least 98%, and particularly preferably at least 99%. The homology or identity of the base sequence may be less than 100%. The homology or identity between base sequences can be determined using known algorithms, such as BLAST. The nucleic acid molecule encompasses a nucleic acid molecule containing a base sequence having at least 90% identity with the base sequence of SEQ ID NO: 1 enabling the parasitic dinoflagellates of the genus Amoebophrya having the nucleic acid molecule to have the ability to parasitize dinoflagellates of the genus Karenia.

In (4), (8), and (12) above, the phrase “stringent conditions” refers to conditions under which only specific hybridization occurs and non-specific hybridization does not occur. An example of such conditions is hybridization in 1×SSC (0.9 M NaCl, 0.09 M trisodium citrate) or 6×SSPE (3M NaCl, 0.2M NaH₂PO₄, 20 mM EDTA·2Na, pH 7.4) at 42° C., followed by washing with 0.5×SSC at 42° C. However, stringent conditions are not limited to this example. Such conditions are described, for example, in M. R. Green et al., Molecular Cloning: A Laboratory Manual, Fourth Edition, Cold Spring Harbor Laboratory Press (2012).

In (5) above, the nucleic acid molecule encompasses a nucleic acid molecule containing the base sequence of SEQ ID NO: 2 enabling the parasitic dinoflagellates of the genus Amoebophrya having the nucleic acid molecule to have the ability to parasitize dinoflagellates of the genus Karenia.

In (6) above, the number of one or several bases to be substituted, deleted, added, or inserted is not particularly limited as long as the number is an integer of 1 or more. For example, the number of one or several bases may be about 1 to several dozen, preferably about 1 to 30, more preferably about 1 to 15, even more preferably about 1 to 10, and particularly preferably about 1 to 5. The nucleic acid molecule encompasses a nucleic acid molecule containing a base sequence having one or several bases substituted, added, or deleted in the base sequence of SEQ ID NO: 2 enabling the parasitic dinoflagellates of the genus Amoebophrya having the nucleic acid molecule to have the ability to parasitize dinoflagellates of the genus Karenia.

In an aspect, the number of one or more bases to be substituted, added, or deleted may be about 1 to 76 so that the identity of the base sequence with the base sequence of SEQ ID NO: 2 is at least 90%, preferably about 1 to 38 so that the identity is at least 95%, more preferably about 1 to 15 so that the identity is at least 98%, and particularly preferably about 1 to 8 so that the identity is at least 99%.

In (7) above, the identity of the base sequence may be at least 90%, preferably at least 95%, more preferably at least 98%, and particularly preferably at least 99%. The homology or identity between the base sequences may be less than 100%. The homology or identity between base sequences can be determined using known algorithms, such as BLAST. The nucleic acid molecule encompasses a nucleic acid molecule containing a base sequence having at least 90% identity with the base sequence of SEQ ID NO: 2 enabling the parasitic dinoflagellates of the genus Amoebophrya having the nucleic acid molecule to have the ability to parasitize dinoflagellates of the genus Karenia.

In (9) above, the nucleic acid molecule encompasses a nucleic acid molecule containing the base sequence of SEQ ID NO: 3 enabling the parasitic dinoflagellates of the genus Amoebophrya having the nucleic acid molecule to have the ability to parasitize dinoflagellates of the genus Karenia.

In (10) above, the number of one or several bases to be substituted, deleted, added, or inserted is not particularly limited as long as the number is an integer of 1 or more. For example, the number of one or several bases may be about 1 to several dozen, preferably about 1 to 30, more preferably about 1 to 15, even more preferably about 1 to 10, and particularly preferably about 1 to 5. The nucleic acid molecule encompasses a nucleic acid molecule containing a base sequence having one or several bases substituted, added, or deleted in the base sequence of SEQ ID NO: 3 enabling the parasitic dinoflagellates of the genus Amoebophrya having the nucleic acid molecule to have the ability to parasitize dinoflagellates of the genus Karenia.

In an aspect, the number of one or more bases to be substituted, added, or deleted may be about 1 to 76 so that the identity of the base sequence with the base sequence of SEQ ID NO: 3 is at least 90%, preferably about 1 to 38 so that the identity is at least 95%, more preferably about 1 to 15 so that the identity is at least 98%, and particularly preferably about 1 to 8 so that the identity is at least 99%.

In (11) above, the identity of the base sequence may be at least 90%, preferably at least 95%, more preferably at least 98%, and particularly preferably at least 99%. The homology or identity between the base sequences may be less than 100%. The homology or identity between base sequences can be determined using known algorithms, such as BLAST. The nucleic acid molecule encompasses a nucleic acid molecule containing a base sequence having at least 90% identity with the base sequence of SEQ ID NO: 3 enabling the parasitic dinoflagellates of the genus Amoebophrya having the nucleic acid molecule to have the ability to parasitize dinoflagellates of the genus Karenia.

In the parasitic dinoflagellates of the genus Amoebophrya having any of the nucleic acid molecules (1) to (4) of the present invention, it is preferred that the nucleic acid molecule also satisfies any of (5) to (8).

Examples of such parasitic dinoflagellates of the genus Amoebophrya include Amoebophrya sp. OSK2020 and Amoebophrya sp. HMN2020. Amoebophrya sp. OSK2020 has a nucleic acid molecule containing the base sequence of SEQ ID NO: 1 (18S rDNA, 1742 bases) and the base sequence of SEQ ID NO: 2: (28S rDNA, 759 bases). Amoebophrya sp. HMN2020 has a nucleic acid molecule containing the base sequence of SEQ ID NO: 3 (28S rDNA, 760 bases).

The culture of the parasitic dinoflagellates of the genus Amoebophrya of the present invention can be performed by, but is not limited to, for example, co-culturing with dinoflagellates of the genus Karenia.

When the method for culturing the parasitic dinoflagellates of the genus Amoebophrya of the present invention includes the step of co-culturing with dinoflagellates of the genus Karenia, the parasitic dinoflagellates of the genus Amoebophrya can be cultured, for example, according to the following procedure.
(a) Inoculate the parasitic dinoflagellates of the genus Amoebophrya of the present invention into a medium in which dinoflagellates of the genus Karenia that are not infected (uninfected) with the parasitic dinoflagellates of the genus Amoebophrya are cultured.
(b) Co-culture the parasitic dinoflagellates of the genus Amoebophrya with dinoflagellates of the genus Karenia.
(c) After two or more days from inoculation (after confirming the spread of infection with a microscope), inoculate a portion of the medium containing the infected host and/or the parasitic dinoflagellates of the genus Amoebophrya alone in step (b) into a fresh medium in which uninfected dinoflagellates of the genus Karenia are cultured.
(d) Repeat steps (b) to (c).

In steps (a) to (d), the medium used for co-culture is preferably a commonly used medium for marine microalgae. A specific example is Daigo's IMK medium (manufactured by Nihon Pharmaceutical Co., Ltd.).

In step (a), inoculation is preferably performed with the concentration of the dinoflagellates of the genus Karenia being 1000 to 2000 cells mL⁻¹ and the concentration of the parasitic dinoflagellates of the genus Amoebophrya being 10000 to 20000 cells mL⁻¹, such that the ratio of the concentration of the parasitic dinoflagellates of the genus Amoebophrya to the concentration of the dinoflagellates of the genus Karenia falls within the range of 1:1 to 1:10.

In step (b), the co-culture is preferably performed under the following conditions: a temperature of 16 to 25° C. (preferably 18 to 24° C.), a light intensity of 50 to 200 μmol photons m⁻² s⁻¹, and a light and dark cycle of 12 hours of light and 12 hours of darkness to 14 hours of light and 10 hours of darkness.

In step (c), replanting of parasitic dinoflagellates of the genus Amoebophrya from the medium is preferably performed 2 to 10 days after inoculation, and more preferably 3 to 4 days after inoculation. Replanting within a period of 2 to 10 days after inoculation is easy and preferred because parasitic dinoflagellates of the genus Amoebophrya would have proliferated in the dinoflagellates of the genus Karenia (host) and then have destroyed the host and come out. The parasitic dinoflagellates of the genus Amoebophrya of the present invention will die in about 3 to 7 days if they do not parasitize the host. Thus, if the operation of step (c) is not performed for 3 to 7 days or more with few new host cells present after the parasitic dinoflagellates of the genus Amoebophrya destroy the host and emerge from the host cells, the parasitic dinoflagellates of the genus Amoebophrya may not be able to parasitize the host and die.

In step (b) above, after the infection of the parasitic dinoflagellates of the genus Amoebophrya with the host is confirmed at a temperature of 16 to 25° C. and a light intensity of 50 to 200 μmol photons m⁻² s⁻¹ with a light and dark cycle of 12 hours of light and 12 hours of darkness to 14 hours of light and 10 hours of darkness, and before the host is destroyed and emerged (for example, 48 to 120 hours after inoculation), the temperature is lowered, preferably to 10 to 15° C., more preferably to 12 to 15° C. Storage of the co-cultures at the lower temperature can delay the infection rate or stop the infection of parasitic dinoflagellates of the genus Amoebophrya with dinoflagellates of the genus Karenia, compared to incubation at a temperature of 16 to 25° C. Storage in the temperature range described above is preferred because it can store parasitic dinoflagellates of the genus Amoebophrya for a longer period of time in a state in which dinoflagellates of the genus Karenia are infected with parasitic dinoflagellates of the genus Amoebophrya. This is probably because creating a low-temperature environment with a parasite proliferating in the host cells temporarily delays or ends the infection cycle and maintains the state in which the host holds the parasite in its cell.

In this case, the infection of uninfected dinoflagellates of the genus Karenia can be confirmed by storing the co-cultures at a temperature of 10 to 15° C. for preferably 15 to 40 days, more preferably for 20 to 40 days, and particularly preferably for 25 to 35 days, then returning the temperature to 16 to 25° C., and then performing step (C).

Further, in step (b) above, after the infection of the parasitic dinoflagellates of the genus Amoebophrya with the host is confirmed at a temperature of 16 to 25° C. and a light intensity of 50 to 200 μmol photons m⁻² s⁻¹ with a light and dark cycle of 12 hours of light and 12 hours of darkness to 14 hours of light and 10 hours of darkness, and before the host is destroyed and emerged (for example, 48 to 120 hours after inoculation), the light intensity is decreased, preferably at 5 to 40 μmol photons m⁻² s⁻¹, more preferably 5 to 20 μmol photons m⁻² s⁻¹, and particularly preferably 5 to 10 μmol photons m⁻² s⁻¹. Storage of the co-cultures at the lower light intensity can delay the infection rate or stop the infection of parasitic dinoflagellates of the genus Amoebophrya with dinoflagellates of the genus Karenia, compared with culture at a light intensity of 5 to 200 μmol photons m⁻² s⁻¹. Storage in the light intensity range described above is preferred because it can store parasitic dinoflagellates of the genus Amoebophrya for a longer period of time in a state in which dinoflagellates of the genus Karenia are infected with parasitic dinoflagellates of the genus Amoebophrya. This is probably because creating a low-light intensity environment with a parasite proliferating in the host cell reduces the activity of the parasite and does not allow the parasite to mature in the host cell.

In this case, the infection of uninfected dinoflagellates of the genus Karenia can be confirmed by storing the co-cultures at a light intensity of 5 to 40 μmol photons m⁻²s⁻¹ for preferably 15 to 100 days, more preferably 25 to 100 days, and particularly preferably 35 to 90 days, then returning the light intensity to 50 to 200 μmol photons m⁻² s⁻¹, and then performing step (C).

Further, in step (b) above, after the infection of the parasitic dinoflagellates of the genus Amoebophrya with the host is confirmed at a temperature of 16 to 25° C. and a light intensity of 50 to 200 μmol photons m⁻² s⁻¹ with a light and dark cycle of 12 hours of light and 12 hours of darkness to 14 hours of light and 10 hours of darkness, and before the host is destroyed and emerged (for example, 48 to 120 hours after inoculation), storage of the co-cultures may be performed by decreasing the temperature and light intensity with a combination of the low temperature conditions and low light intensity conditions described above.

In this case as well, in regards to the infection of dinoflagellates of the genus Karenia with parasitic dinoflagellates of the genus Amoebophrya, this operation can delay the infection rate or end the infection as compared with a culture at a temperature of 16 to 25° C. and a light intensity of 50 to 200 μmol photons m⁻² s⁻¹.

Culture of dinoflagellates of the genus Karenia is preferably performed under the following conditions: a temperature of 20 to 25° C. and a light intensity of 50 to 100 μmol photons m⁻² s⁻¹ with a light and dark cycle of 14 hours of light and 10 hours of darkness, regardless of whether the culture is before or after infection with the parasitic dinoflagellates of the genus Amoebophrya of the present invention.

Examples of the parasitic dinoflagellates of the genus Amoebophrya of the present invention include Amoebophrya sp. OSK2020. Amoebophrya sp. OSK2020 is preserved and maintained by the present Applicant, and the Applicant guarantees the distribution of Amoebophrya sp. OSK2020 according to the provisions of Article 27-3 of the Ordinance for Enforcement of the Patent Act of Japan, provided that the laws are complied with.

Amoebophrya sp. OSK2020 is a parasitic dinoflagellate of the genus Amoebophrya obtained from Karenia mikimotoi, a dinoflagellate of the genus Karenia in a seawater sample collected from Osaka Bay. Amoebophrya sp. OSK2020 has a nucleic acid molecule containing the base sequence of SEQ ID NO: 1 (18S rDNA, 1742 bases) and the base sequence of SEQ ID NO: 2 (28S rDNA, 759 bases). The method for obtaining Amoebophrya sp. OSK2020 is, for example, as follows.
(i) Observe a large amount of Karenia mikimotoi that have grown in the seawater at a site with a fluorescence microscope. Karenia mikimotoi infected with Amoebophrya sp. contains strong green fluorescence (derived from Amoebophrya) in the cells, so the infected Karenia mikimotoi is removed and inoculated into a medium containing uninfected Karenia mikimotoi that has been cultured beforehand.
(ii) Incubate the cells for about several days to 1 week after inoculation (temperature: 24° C., light and dark cycle: 14 hours of light and 10 hours of darkness (light period: white fluorescent lamp of 100 μmol photons m⁻² s⁻¹)).
(iii) Confirm the spread of the infection with Amoebophrya sp. with a fluorescence microscope.

Further, the method for culturing Amoebophrya sp. OSK2020 is, for example, as follows. Amoebophrya sp. OSK2020 can be stably maintained according to the following culture method.
(iv) Inoculate a portion of the medium containing Karenia mikimotoi cells infected with Amoebophrya sp. OSK2020 into a medium in which uninfected Karenia mikimotoi is cultured.
(v) After inoculation, perform culture for 3 to 5 days, preferably 3 to 4 days (temperature: 16 to 25° C. (preferably 18 to 24° C.), light and dark cycle: 14 hours of light and 10 hours of darkness (light period: white fluorescent lamp of 100 μmol photons m⁻² s⁻¹)).
(vi) Confirm the spread of the infection with Amoebophrya sp. OSK2020 with a fluorescence microscope.
(vii) Repeat steps (iv) to (vi).

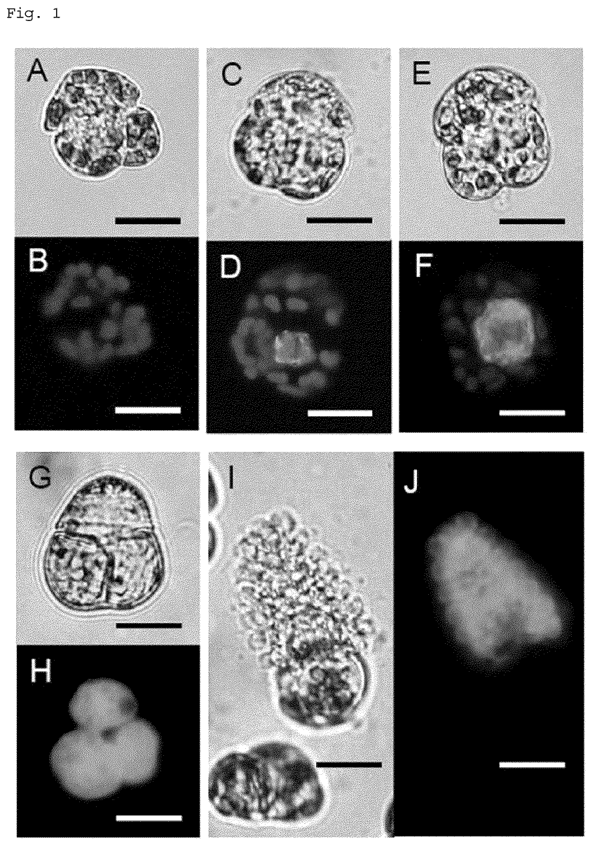

FIG. 1 shows how Amoebophrya sp. OSK2020 parasitizes Karenia mikimotoi, a dinoflagellate of the genus Karenia (the scale bars all indicating 20 μm). FIGS. 1A and 1B show uninfected Karenia mikimotoi. FIGS. 1C and 1D show Karenia mikimotoi in the early stage of infection. Amoebophrya sp. OSK2020 grow to several dozen in a cell of the host, Karenia mikimotoi. FIGS. 1E and 1F show the middle stage of infection, and FIGS. 1G and 1H show the late stage of infection. Amocbophrya sp. OSK2020, which has grown to hundreds in the cell of the host, Karenia mikimotoi, eventually breaks through the cell of Karenia mikimotoi and exits the cell (FIGS. 1I and 1J). Thereafter, hundreds of Amoebophrya sp. OSK2020 then disperse into water and parasitize new hosts. This series of cycle is completed in 3 to 5 days, and preferably 3 to 4 days.

In step (v) above, after the infection of Amoebophrya sp. OSK2020 with Karenia mikimotoi is confirmed at a temperature of 16 to 25° C. and a light intensity of 100 μmol photons m⁻² s⁻¹ with a light and dark cycle of 14 hours of light and 10 hours of darkness, and before Karenia mikimotoi is destroyed and emerged (for example, 48 to 120 hours after inoculation), the temperature is lowered, preferably to 10 to 15° C., more preferably to 12 to 15° C. Storage of the co-cultures at the lower temperature can delay the infection rate or stop the infection of Amoebophrya sp. OSK2020 with Karenia mikimotoi, compared to incubation at a temperature of 16 to 25° C. Storage in the temperature range described above is preferred because it can store Amoebophrya sp. OSK2020 for a longer period of time in a state in which Karenia mikimotoi is infected with Amoebophrya sp. OSK2020.

In this case, the infection of uninfected Karenia mikimotoi can be confirmed by storing the co-cultures at a temperature of 10 to 15° C. for preferably 15 to 40 days, more preferably for 20 to 40 days, and particularly preferably for 25 to 35 days, then returning the temperature to 16 to 25° C., and then performing step (iv) in step (vii).

Further, in step (v) above, after the infection of Amoebophrya sp. OSK2020 with Karenia mikimotoi is confirmed at a temperature of 16 to 25° C. and a light intensity of 100 μmol photons m⁻² s⁻¹ with a light and dark cycle of 14 hours of light and 10 hours of darkness, and before Karenia mikimotoi is destroyed and emerged (for example, 48 to 120 hours after inoculation), the light intensity is decreased, preferably at 5 to 40 μmol photons m⁻² s⁻¹, more preferably 5 to 20 μmol photons m⁻² s⁻¹, and particularly preferably 5 to 10 μmol photons m⁻² s⁻¹. Storage of the co-cultures at the lower light intensity can delay the infection rate or stop the infection of Amoebophrya sp. OSK2020 with Karenia mikimotoi, compared with culture at a light intensity of 5 to 200 μmol photons m⁻² s⁻¹. Storage in the light intensity range described above is preferred because it can store Amoebophrya sp. OSK2020 for a longer period of time in a state in which Karenia mikimotoi is infected with Amoebophrya sp. OSK2020.

In this case, the infection of uninfected Karenia mikimotoi can be confirmed by storing the co-cultures at a light intensity of 5 to 40 μmol photons m⁻² s⁻¹ for preferably 15 to 100 days, more preferably 25 to 100 days, and particularly preferably 35 to 90 days, then returning the light intensity to 50 to 200 μmol photons m⁻² s⁻¹, and then performing step (iv) in (vii).

Alternatively, in step (v) above, after the infection of Amoebophrya sp. OSK2020 with Karenia mikimotoi is confirmed at a temperature of 16 to 25° C. and a light intensity of 100 μmol photons m⁻² s⁻¹ with a light and dark cycle of 14 hours of light and 10 hours of darkness, and before Karenia mikimotoi is destroyed and emerged (for example, 48 to 120 hours after inoculation), storage of the co-cultures may be performed by decreasing the temperature and light intensity with a combination of the low temperature conditions and low light intensity conditions described above. In this case as well, in regards to the infection of Karenia mikimotoi with Amoebophrya sp. OSK2020, this operation can delay the infection rate or end the infection as compared with culture at a temperature of 16 to 25° C. and a light intensity of 50 to 200 μmol photons m⁻² s⁻¹.

In another aspect, the present invention provides a composition containing the parasitic dinoflagellates of the genus Amoebophrya of the present invention. The composition can be used in the prevention of red tide. More preferably, the composition can be used for red tide derived from the growth of dinoflagellates of the genus Karenia.

In another aspect, the present invention also provides a microbial pesticide containing the parasitic dinoflagellates of the genus Amoebophrya of the present invention. The microbial pesticide is suitable for the prevention of red tide, particularly for the prevention of red tide derived from the growth of dinoflagellates of the genus Karenia.

For the composition or microbial pesticide of the present invention, a culture solution, in-situ seawater, or in-situ seabed mud containing the parasitic dinoflagellates of the genus Amoebophrya may be used as is. However, the composition or microbial pesticide may also contain additives, such as a stabilizer and glycerol, to the extent that the effects of the present invention are not impaired.

The composition or microbial pesticide of the present invention can be produced according to, for example, a method including the step of culturing the parasitic dinoflagellates of the genus Amoebophrya of the present invention. The step of culturing the parasitic dinoflagellates of the genus Amoebophrya of the present invention may be the method for co-culturing parasitic dinoflagellates of the genus Amoebophrya with dinoflagellates of the genus Karenia described above.

In another aspect, the present invention also provides a method for preventing red tide. The composition or microbial pesticide of the present invention is sprayed over the sea surface or into the sea to allow the parasitic dinoflagellates of the genus Amoebophrya of the present invention contained in the composition or microbial pesticide of the present invention to infect the dinoflagellates of the genus Karenia present on the sea surface or in the sea. This kills the dinoflagellates of the genus Karenia present on the sea surface or in the sea, inhibits the growth of the dinoflagellates of the genus Karenia, and prevents red tide derived from the growth of the dinoflagellates of the genus Karenia. The amount of the composition or microbial pesticide of the present invention sprayed over the sea surface or into the sea is not particularly limited, and is appropriately determined according to the area of the sea surface to be sprayed, the depth of the sea, and the like.

In a preferred embodiment, the parasitic dinoflagellates of the genus Amoebophrya of the present invention are expected to have a small impact on the ecosystem and be highly safe, as the parasitic dinoflagellates die in about 7 days if they cannot parasitize the host, i.e., dinoflagellates of the genus Karenia, due to their absence. Additionally, if the parasitic dinoflagellates of the genus Amoebophrya of the present invention specifically infect dinoflagellates of the genus Karenia, there is little concern about infection of non-specific, many organisms; thus, the parasitic dinoflagellates of the genus Amoebophrya are expected to be highly safe.

### EXAMPLES

The present invention is described in more detail with reference to Examples below. However, the present invention is not limited to these Examples.

### Acquisition of Amoebophrya Sp. OSK2020, Parasitic Dinoflagellate of Genus Amoebophrya

Karenia mikimotoi (a dinoflagellate of the genus Karenia) infected with Amoebophrya sp. (a parasitic dinoflagellate of the genus Amoebophrya) was obtained from a seawater sample (water temperature: 24° C., salt content: 30) collected from Osaka Bay on Oct. 5, 2020. Infected individuals of Karenia mikimotoi were inoculated into a medium in which uninfected Karenia mikimotoi (a strain collected from Ago Bay in 2013, stored in Daigo's IMK medium with a salt content of 30) was cultured. After inoculation, culture was performed for 4 days (temperature: 24° C., light and dark cycle: 14 hours of light and 10 hours of darkness (light period: white fluorescent lamp of 100 μmol photons m⁻² s⁻¹)). After the infection with Amoebophrya sp. spread to uninfected Karenia mikimotoi, about 1/10 to 1/20 of the medium (including the infected host) was aspirated with a micropipette and inoculated into a fresh medium (containing an uninfected host), thereby obtaining Amoebophrya sp. OSK2020 (which may sometimes be referred to as “OSK2020” below). According to this method, strains other than OSK2020 can also be obtained by performing similar operations in other bodies of water in which Karenia mikimotoi grows.

The stock culture of OSK2020 was performed by inoculating every 4 days a predetermined amount of Karenia mikimotoi infected with OSK2020 into a medium in which uninfected Karenia mikimotoi was cultured. The stock culture was performed under the following conditions: a temperature of 24° C., and a light and dark cycle of 14 hours of light and 10 hours of darkness (light period: white fluorescent lamp of 100 μmol photons m⁻² s⁻¹). With this culture method, OSK2020 was successfully maintained stably for more than one year.

### Example 1: Experiment on Infection of Karenia Mikimotoi With OSK2020

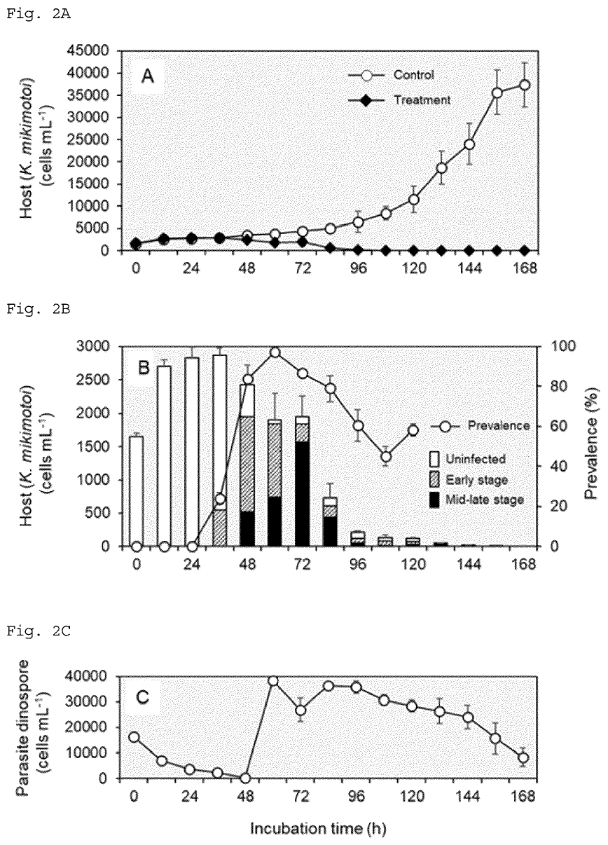

An experiment of infection was conducted at the laboratory level to confirm the relationship between the cell density of Karenia mikimotoi, which is the host of OSK2020, and the parasitic effects of OSK2020. A culture medium of OSK2020 was passed through a filter with a pore size of 8 μm (Nuclepore Track-Etched Membrane, manufactured by Whatman) to remove the host cells, thereby isolating migratory cells of OSK2020. 20 mL of the migratory cells within 12 hours since the emergence from the host were inoculated at an initial concentration of 16,000 cells mL⁻¹ into three-time passaged Karenia mikimotoi (initial cell concentration: 1,600 cells mL⁻¹) in the logarithmic growth phase, and cultured under the conditions described above. FIG. 2 shows the results.

FIG. 2A shows changes in cell density of Karenia mikimotoi over time when OSK2020 was added to the culture medium of Karenia mikimotoi at a point of 0 hours in time from the start of culture (treatment) and when OSK2020 was not added (control). FIG. 2B shows the infection rate and infection state over time of Karenia mikimotoi when OSK2020 was added to the culture medium of Karenia mikimotoi at a point of 0 hours in time from the start of culture. FIG. 2C shows change in cell density of OSK2020 over time when OSK2020 was added to the culture medium of Karenia mikimotoi at a point of 0 hours in time from the start of culture. From these results, it is clear that when OSK2020 is added, Karenia mikimotoi is infected with OSK2020 and killed.

### Example 2: Experiment on Infection of Other Microorganisms With OSK2020

To confirm what can be the host of OSK2020, a cross-infection experiment was conducted. The following 16 species of microorganisms were examined. The experiment was conducted according to Kim, J. Phycol. 2006, 42, 1170-1173; Chambouvet et al., Protist 2011, 162, 637-649; Chen et al., J. Eukaryot. Microbiol. 2018, 65, 448-457; and Nishitani et al. Harmful Algae 2021, 110, 102123. The operation for isolating OSK2020 migratory cells was performed in the same manner as in Example 1. The microorganisms under study were cultured to achieve an appropriate cell density, and then a culture medium containing an individual microorganism under study was added in an amount of 2 mL each to a 24-well plate. Thereafter, migratory cells of OSK2020 were added to achieve a ratio of the migratory cells to the host microorganism (migratory cells: host microorganism) of 20:1, and the mixture was cultured at a culture temperature of 20° C. and a light and dark cycle of 14:10 (light period: white fluorescent lump of 100 μmol photons m⁻² s⁻¹). The green autofluorescence of OSK2020 and the red autofluorescence of chloroplasts of the host microorganism were observed with a fluorescence microscope (IX71, manufactured by Olympus Corporation) equipped with a U-MNBV2 cube fluorescence mirror unit (excitation filter: 420 to 440 nm, absorption filter: 475 nm, manufactured by Olympus Corporation) to confirm the parasitism.

### Microorganisms Studied

Akashiwo sanguinea
A. catenella (Group I)
A. pacificum
Cochlodinium polykrikoides
Gonyaulax polygramma
Gonyaulax spinifera
Gymnodinium impudicum
Heterocapsa circularisquama
Karenia mikimotoi
Karenia papilionacea
Levanderina fissa
Prorocentrum koreanum
Prorocentrum minimum
Prorocentrum mexicanum
Scrippsiella trochoidea
Takayama xiamenensis

Table 1 shows the results. OSK2020 infected only Karenia mikimotoi and Karenia papilionacea, and infected no other microorganisms. This revealed that OSK2020 parasitizes only dinoflagellates of the genus Karenia and does not affect other organisms.

TABLE 1

| Microorganisms Infected with | Microorganisms Not Infected with |
|---|---|
| OSK2020 | OSK2020 |
| Karenia mikimotoi | Akashiwo sanguinea |
| Karenia papilionacea | Alexandrium catenella |
| | Alexandrium pacificum |
| | Cochlodinium polykrikoides |
| | Gonyaulax polygramma |
| | Gonyaulax spinifera |
| | Gymnodinium impudicum |
| | Takayama xiamenensis |
| | Heterocapsa circularisqama |
| | Levandarina fissa |
| | Prorocentrum koreanum |
| | Prorocentrum mimimum |
| | Prorocentrum mexicanum |
| | Scrippsiella trochoidea |

### Example 3: Extraction of DNA of Amoebophrya sp. 20200ct5-1 to 5 From Seawater Sample

To confirm the genetic diversity of the parasitic dinoflagellates of the genus Amoebophrya with the ability to parasitize Karenia mikimotoi, five types of infected Karenia mikimotoi (20200ct5-1, 20200ct5-2, 20200ct5-3, 20200ct5-4, and 20200ct5-5) were collected according to a micropipette aspiration technique from seawater samples in which OSK2020 was found. Each type of cells was washed at least three times with seawater disinfected with a filter (pore size: 0.1 μm) and then placed in a 0.2 mL PCR tube, together with a 5% Chelex® suspension (Bio-Rad Laboratories, Hercules). In accordance with the method described in Richlen and Barber Mol. Ecol. Notes 2005, 5, 688-691, DNA of Amoebophrya sp. 20200ct5-1 to 20200ct5-5 was extracted.

### Example 4: Extraction of DNA of Amoebophrya sp. HMN2020 From Seawater Sample

Karenia mikimotoi confirmed to have been parasitized by Amoebophrya was collected from lake water samples collected at Lake Hamana on Dec. 10, 2020, according to a micropipette aspiration technique. The cells were washed at least three times with seawater sterilized with a filter (pore size: 0.1 μm) and then placed in a 0.2 mL PCR tube, together with a 5% Chelex® suspension (manufactured by Bio-Rad Laboratories, Hercules). In accordance with the method described in Richlen and Barber Mol. Ecol. Notes 2005, 5, 688-691, DNA of Amoebophrya sp. HMN2020 was extracted.

### Example 5: Sequence Determination

RCR of nucleus 18S rDNA and nucleus 28S rDNA of OSK2020 and nucleus 28S rDNA obtained in Examples 3 and 4 was performed. The sequence of 18S rDNA was determined according to the method described in Nishitani et al., Appl Environ Microbiol., 2010, 76, 2791-2798, and the sequence of nucleus 28S rDNA was determined according to the method described in Nishitani et al., Harmful Algae 2021, 110, 102123. Specifically, 18S rDNA and 28S rDNA from the extracted DNA of the genus Amoebophrya were amplified by using the TOYOBO KOD plus ver. 2 enzyme with primers. The product obtained after completion of PCR was confirmed by performing 1.5% TAE agarose gel electrophoresis, purified by Exo SAP IT (78200, Affymetrix, California), and then subjected to direct sequencing to determine the sequence.

The obtained sequence of nucleus 18S rDNA of Amoebophrya sp. OSK2020 is represented by SEQ ID NO: 1. The obtained sequence of nucleus 28S rDNA of Amoebophrya sp. OSK2020 is represented by SEQ ID NO: 2. The obtained sequence of nucleus 28S rDNA of Amoebophrya sp. 20200ct5-1 is represented by SEQ ID NO: 4. The obtained sequences of nucleus 28S rDNA of Amoebophrya sp. 20200ct5-2, Amoebophrya sp. 20200ct5-3, Amoebophrya sp. 20200ct5-4, and Amoebophrya sp. 20200ct5-5 are represented by SEQ ID NO: 2. The obtained sequence of nucleus 28S rDNA of Amoebophrya sp. HMN2020 is represented by SEQ ID NO: 3.

### Example 6: Phylogenetic Analysis

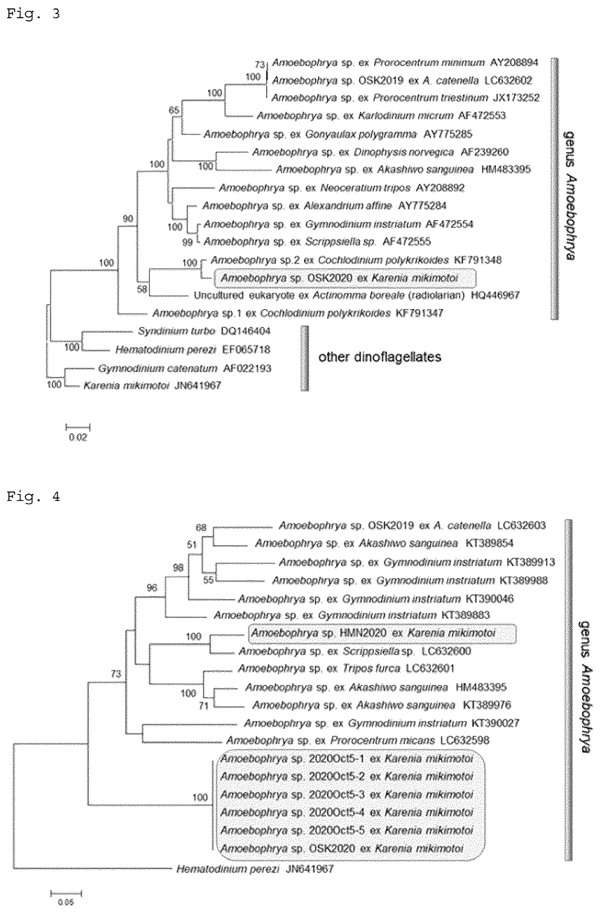

The sequence of the genus Amoebophrya obtained in Example 5 was confirmed using BLAST with GenBank, and phylogenetic trees of nucleus 18S rDNA and nucleus 28S rDNA were created using MEGA ver. X software (Kumar et al., Mol. Biol. Evol. 2018, 35, 1547-1549) according to the maximum likelihood method. The topology of the phylogenetic trees was evaluated by bootstrapping with 100 repetitions. FIGS. 3 and 4 show the results.

FIG. 3 shows the results of molecular phylogenetic analysis of parasitic organisms according to nucleus 18S rDNA. The sequence of Amoebophrya sp. OSK2020 enclosed in a rectangular frame in FIG. 3 is represented by SEQ ID NO: 1. FIG. 4 shows the results of molecular phylogenetic analysis of parasitic organisms according to nucleus 28S rDNA. The sequences of Amoebophrya sp. OSK2020 ex. Karenia mikimotoi, and Amoebophrya sp. 20200ct5-2 to 5-5 are represented by SEQ ID NO: 2. The sequence of Amoebophrya sp. HMN2020 ex. Karenia mikimotoi is represented by SEQ ID NO: 3. The sequences of Amoebophrya sp. 20200ct5-1 in FIG. 4 is represented by SEQ ID NO: 4. The sequences of nucleus 28S rDNA of Amoebophrya sp. 20200ct5-1 differs from the sequence of SEQ ID NO: 2 by only one base.

These genetic analyses revealed that the DNA sequences of Amoebophrya sp. isolated in the present invention did not match any existing sequence registered in the database. In other words, it suggests that this Amoebophrya sp. may be a completely new species that has never been isolated anywhere in the world before. Currently, environmental DNA, including dinoflagellates, is being vigorously investigated in maritime areas around the world, but the Amoebophrya sp. did not match any of them. This suggests that the novel species, Amoebophrya sp. OSK2020, of the present invention is confined to Osaka Bay or nearby bodies of water in Japan.

### Example 7: Low-temperature Storage Experiment on Infected Host Cell

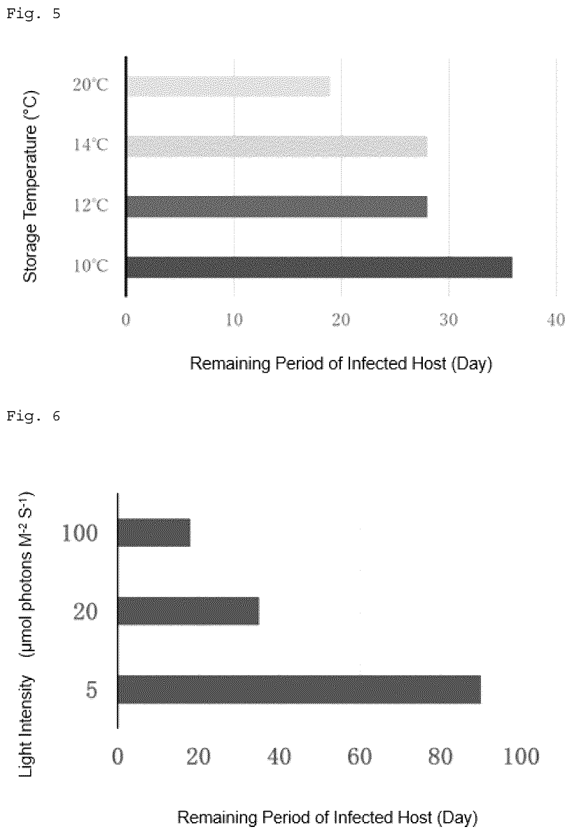

Daigo's IMK medium, a host (Karenia mikimotoi) (2,000 cells/mL), and a parasite (Amoebophrya sp. OSK2020) (20,000 cells/mL) were individually added to the wells of a 12-well microplate and cultured at a water temperature of 20° C. (four microplates were prepared). When the infection rate reached about 70% (this was determined to be day 0), the microplates were transferred to the environment of 20° C. (control), 14° C., 12° C., and 10° C., followed by recording the days of the infected host remained in cold storage (the number of days until complete extinction) (FIG. 5). The light intensity was set to 100 μmol photons m⁻² s⁻¹.

Thereafter, whether the halted infection cycle had resumed was confirmed. The infected hosts that had been stored at low temperatures were supplemented with an uninfected host, and the water temperature was returned to 20° C. Whether the halted infection cycle resumed and caused reinfection was investigated every week.

In the control experimental group at 20° C. (optimum temperature), the infected host remained for 19 days. However, in the experimental groups, for which the infection state was temporarily suspended at low temperatures of 14° C. and 12° C., the number of days the infected host of each group remained was extended to 28 days. In the experimental group placed at 10° C., the number of days the infected host remained was extended up to 36 days. It is thought that when placed at low temperatures, the parasite diminished its activity and was unable to mature within the host cells. Moreover, an attempt to resume the infection cycle, which had been temporarily halted, resulted in successful reinfection from the infected hosts stored at 14° C. for 19 days, 12° C. for 28 days, and 10° C. for 36 days, compared with the control stored at 20° C. for 7 days. Although the current sub-culturing interval of the host (Karenia mikimotoi) and the parasite (Amoebophrya sp. OSK2020) is as short as about 3 to 5 days, storing the infected host at 10°C. was found to be able to extend this period to about one month. Because the host cells could not withstand the cold at temperatures lower than 10° C. (e.g., 8° C.), at which the host and the parasite were both completely killed, the optimal storage temperature is expected to be 10 to 12° C.

Detailed consideration of storage conditions may make it possible to achieve long-term storage of one month or more. These results are considered crucial insights not only for reducing costs and labor but also for producing formulations of parasitic organisms.

### Example 8: Low-Light-Intensity Storage Experiment on Infected Host Cell

In the same manner as in the experiment above, Daigo's IMK medium, a host (Karenia mikimotoi) at 2,000 cells/mL, and a parasite (Amoebophrya sp. OSK2020) at 20,000 cells/mL were added to the wells of a 12-well microplate and were cultured at a water temperature of 20° C. (three microplates were prepared). The light intensity was set to 100 μmol photons m⁻² s⁻¹. When the infection rate reached about 70% (this was determined to be day 0), the microplates were transferred to an environment with a light intensity of 100 μmol photons m⁻² s⁻¹ (control), 20 μmol photons m⁻² s⁻¹, and 5 μmol photons m⁻² s⁻¹, followed by recording the number of days the infected host remained in low-light-intensity storage (the number of days until complete extinction) (FIG. 6). The culture temperature during storage was set to 20° C.

Thereafter, whether the halted infection cycle had resumed was confirmed. The infected host that had been stored at low light intensity was supplemented with an uninfected host, and the light intensity was returned to 100 μmol photons m⁻² s⁻¹. Whether the halted infection cycle resumed and caused reinfection was investigated every week.

In the control experimental group with a light intensity of 100 μmol photons m⁻² s⁻¹, which is the light intensity for normal culture, the infected host remained for 18 days. However, in the experimental group, which was placed in a low-light-intensity environment of 20 μmol photons m⁻² s⁻ⁱ to temporarily suspend the infection state, the number of days the infected host remained was extended to 35 days, and the number of days the infected host of the group placed at 5 μmol photons m⁻² s⁻¹ remained was extended to 90 days. It is thought that when placed at a low light intensity, the parasite diminished its activity and was unable to mature within the host cells. Moreover, an attempt to resume the infection cycle, which had been temporarily halted, resulted in successful reinfection from the infected hosts stored for 90 days. Although the current sub-culturing interval of the host (Karenia mikimotoi) and the parasite (Amoebophrya sp. OSK2020) is as short as about 3 to 5 days, storing the infected host at low light intensity was found to be able to extend this period to about three months. At a light intensity lower than 5 μmol photons m⁻² s⁻¹, the host cells, which undergo photosynthesis, could not endure the low-light-intensity conditions, and the host and the parasite were both completely killed. Thus, the optimal light intensity for storage is expected to be 5 to 10 μmol photons m⁻² s⁻¹.

1. A composition comprising a parasitic dinoflagellate of the genus Amoebophrya, having the ability to parasitize a dinoflagellate of the genus Karenia.

2. A-The composition according to claim 1 comprising a parasitic dinoflagellate of the genus Amoebophrya, comprising one of the following nucleic acid molecules (1) to (12):
(1) a nucleic acid molecule containing the base sequence of SEQ ID NO: 1,
(2) a nucleic acid molecule containing a base sequence having one or several bases substituted, added, or deleted in the base sequence of SEQ ID NO: 1,
(3) a nucleic acid molecule containing a base sequence having at least 90% identity with the base sequence of SEQ ID NO: 1,
(4) a nucleic acid molecule containing a base sequence that hybridizes with a base sequence complementary to the base sequence of SEQ ID NO: 1 under stringent conditions,
(5) a nucleic acid molecule containing the base sequence of SEQ ID NO: 2,
(6) a nucleic acid molecule containing a base sequence having one or several bases substituted, added, or deleted in the base sequence of SEQ ID NO: 2,
(7) a nucleic acid molecule containing a base sequence having at least 90% identity with the base sequence of SEQ ID NO: 2,
(8) a nucleic acid molecule containing a base sequence that hybridizes with a base sequence complementary to the base sequence of SEQ ID NO: 2 under stringent conditions,
(9) a nucleic acid molecule containing the base sequence of SEQ ID NO: 3,
(10) a nucleic acid molecule containing a base sequence having one or several bases substituted, added, or deleted in the base sequence of SEQ ID NO: 3,
(11) a nucleic acid molecule containing a base sequence having at least 90% identity with the base sequence of SEQ ID NO: 3, and
(12) a nucleic acid molecule containing a base sequence that hybridizes with a base sequence complementary to the base sequence of SEQ ID NO: 3 under stringent conditions.

3. The composition according to claim 1, having an ability to kill a dinoflagellate of the genus Karenia.

4. The composition according to claim 1, having an ability to inhibit the proliferation of a dinoflagellate of the genus Karenia.

5. (canceled)

6. The composition according to claim 1, which is for use in the prevention of red tide.

7. A microbial pesticide comprising the composition according to claim 1.

8. A method for obtaining a parasitic dinoflagellate of the genus Amoebophrya, comprising the step of inoculating a dinoflagellate of the genus Karenia infected with a parasitic dinoflagellate of the genus Amoebophrya into a medium in which an uninfected dinoflagellate of the genus Karenia is cultured.

9. (canceled)

10. A method for inhibiting the proliferation of algae of the genus Karenia, comprising the step of allowing a parasitic dinoflagellate of the genus Amoebophrya to parasitize a dinoflagellate of the genus Karenia.
